(19) United States
(12) Patent Application Publication
(10) Pub. No.: US 2026/0364051 A1
(43) Pub. Date: Nov. 19, 2026

(54) ORGANIC SOLVENT FREE DRUG MICROCRYSTALS

(71) Applicant: THE JOHNS HOPKINS UNIVERSITY, Baltimore, MD (US)

(72) Inventors: Laura ENSIGN-HODGES, Baltimore, MD (US); Justin HANES, Baltimore, MD (US); Tung Heng HSUEH, Baltimore, MD (US)

(21) Appl. No.: 19/145,906

(22) PCT Filed: Jan. 4, 2024
(86) PCT No.: PCT/US2024/010255

§ 371 Date: Jul. 4, 2025

Related U.S. Application Data
(60) Provisional application No. 63/478,729, filed on Jan. 6, 2023.

(51) Int. Cl. A61K 31/404; A61K 9/14

(52) U.S. Cl. CPC A61K 31/404; A61K 9/145

(57) ABSTRACT

Administration to the eye, ear or neurological tissue of therapeutic or prophylactic agents that inhibit DLK in micro-crystalline form leads to increased delivery and long-lasting activity as compared with administration of the agent in non-crystalline form. Methods have been developed to form drug microcrystals, especially microcrystals including a tyrosine kinase inhibitor such as sunitinib malate in complex with soluble lipophilic compound containing anions such as pamoic acid, without organic solvent, by precipitation in water. Microcrystals are needle and/or rod shaped.

### CROSS-REFERENCE TO RELATED APPLICATIONS

This application claims the benefit of and priority 5 to U.S.S.N. 63/478,729, filed Jan. 6, 2023, which is incorporated by reference herein in its entirety.

### STATEMENT REGARDING FEDERALLY SPONSORED RESEARCH

This invention was made with government support under grant EY026578 awarded by the National Institutes of Health. The government has certain rights in the invention.

### FIELD OF THE INVENTION

The present invention is in the field of extended-release formulations for ophthalmic administration, and more particularly in the area of injectable formulations of drug microcrystals for sustained drug delivery in the eye, ear, or neurological tissue.

### BACKGROUND OF THE INVENTION

Glaucoma is the leading cause of irreversible blindness, impacting 80 million people worldwide (Quigley, et al., Br J Ophthalmol, (2006) 90(3): 262-7; Fundation, B. Glaucoma: Facts & FIG. 2021). Due to the rapid increase in the aging population, the number is expected to rise to 111 million by 2040 (Tham, Y. C., et al., Ophthalmology (2014) 121(11): 2081-2090). Glaucoma is a neurodegenerative disease that causes irreversible vision loss due to the death of retinal ganglion cells (RGCs). All of the current standard-of-care treatments for glaucoma are directed to lowering intraocular pressure (IOP). However, maintaining lower pressure can be challenging in some patients, and in others, even if significant pressure lowering is achieved, RGC loss continues (Quigley, Lancet (2011) 377(9774): 1367-1377).

Dual leucine zipper kinase (DLK; MAP3K12) is a key mediator of RGC cell death in response in axonal injury (Welsbie, et al., Proc Natl Acad Sci U S A (2013) 110(10): 4045-50; Welsbie, et al., Neuron (2017) 94(6): 1142-1154 e6). Compounds with activity against DLK that potently promote survival of mouse and human RGCs have been identified, including the FDA-approved kinase inhibitor sunitinib (Welsbie, et al., Neuron (2017) 94(6): 1142-1154 e6). Development of sunitinib into a formulation for effective, sustained intraocular delivery as a complement to IOP-lowering strategies could improve glaucoma treatment and management (Chader, Invest Ophthalmol. Vis Sci (2012) 53(5): 2501-5; Chang, Ophthalmology (2012) 119(5): 979-86; Danesh-Meyer, et al. Curr Opin Ophthalmol (2011) 22(2): 78-86; Hanumunthadu, et al. Expert Review of Ophthalmology (2014) 9(2): 109-123).

Patient adherence is critical for treating chronic diseases like glaucoma, though administration difficulties and side effects associated with glaucoma eye drop treatments result in high levels of non-compliance (Kaufman, Ophthalmol Vis Sci (2012) 53(5): 2495-500). Typically, only 40-60% of patients adhere to glaucoma drop therapy regimens, even in cases where the patients were being monitored and were provided free medication (Nordstrom, et al., Am J Ophthalmol (2005) 140(4): 598-606; Okeke, et al., Ophthalmology (2009) 116(12): 2286-93; Okeke, et al., Ophthalmology (2009) 116(2): 191-9).

Achieving effective drug delivery to the posterior segment with an eye drop is challenging (Rodrigues, et al., Pharm Res (2018) 35(12): 245; Singh, et al., Retina (2014) 34(9): 1787-95; Nomoto, et al., Invest Ophthalmol Vis Sci, (2009) 50(10): 4807-13; Iwase, et al., Invest Ophthalmol Vis Sci (2013) 54(1): 503-11). The most common approach for achieving drug delivery to the posterior segment is intravitreal (IVT) injection, which is employed every 6-8 weeks to deliver biologics for treatment of wet age-related macular degeneration (Avery, et al., Ophthalmology (2006) 113(3): 363-372 e5; Becerra, et al., Curr Drug Targets (2011) 12(2): 149-72). However, IVT injection is relatively invasive, and is associated with rare, but severe ocular complications (Jager, et al., Retina (2004) 24(5): 676-98; Boyer, et al., Ophthalmology (2014) 121(10): 1904-14). For a disease that may require treatment for decades, a less invasive injection approach is preferred. Further, longer times between injections would be preferred by glaucoma patients, particularly since it may take years to notice deterioration of vision.

Similar problems arise with treatment of the ear, especially the inner ear, and of neurological tissue such as the brain, spinal cord, and sites of neuroinflammation.

WO2022/221537 published Oct. 20, 2022, describes microcrystals formed of active agents that bind to a receptor or ligand in the eye leads to increased delivery and long-lasting efficacy of the active agent within the eye. Injectable formulations for administration into the eye include one or more active agents which bind to a receptor or ligand in the eye, in the form of microcrystals; and one or more pharmaceutically acceptable excipients for administration to the eye. An exemplary therapeutic agent is an inhibitor of dual leucine zipper kinase (DLK). In preferred embodiments, the active agents bind to a ligand such as melanin within the eye with a dissociation constant (K_{d}) of less than 1×10⁻³ M, 1×10⁻⁴ M, 1×10⁻⁵ M, or 1×10⁻⁶ M. Preferred agents include sunitinib, salts, derivatives, analogues, or prodrugs thereof. In a preferred embodiment, the microcrystals are formed of a complex between sunitinib malate and pamoic acid. Typically, the size of the microcrystals is between about 2 microns and about 200 microns, preferably between about 5 microns and about 100 microns, and the pH of the formulation is between about pH 6 and about pH 8. In some embodiments, the microcrystals are formulated for injection, for example, by intravitreal, subconjunctival, or suprachoroidal administration. However, in all cases the microcrystals are made using organic solvents, which are extremely difficult to remove completely and therefore present potential toxicity issues in confined areas such as the eye and inner ear.

The size and shape of the microcrystals are critical for effective delivery into the eye. It is also important to avoid or minimize the presence of organic solvent not only for compliance with the requirements of the Food and Drug Administration but to avoid toxicity in the confined small spaces of the eye into which the drugs are administered.

Therefore, it is an object of the invention to provide formulations for the effective and sustained intraocular delivery of active agents to treat or alleviate one or more symptoms of glaucoma and to provide formulations for the effective and sustained delivery of active agents to the posterior segment of the eye, which are made without organic solvents.

### SUMMARY OF THE INVENTION

Methods to make microcrystals of defined size and shape without organic solvent have been developed. The methods also have the benefit of increasing the drug concentration and facilitate stability after lyophilization and reconstitution. These methods for making organic solvent free formulations facilitate injection of the microcrystals in small volumes, for example, for delivery to the eye, the inner ear, and the spinal cord and brain for neuroprotection.

In the preferred embodiment, the microcrystals are formed of an inhibitor of dual leucine zipper kinase (DLK) such as sunitinib, salt, analogue, or prodrug thereof. In the most preferred embodiment, the microcrystals are formed by mixing sunitinib salt such as sunitinib malate with pamoic acid and precipitating in water as a function of the ratio of sunitinib salt with pamoic acid. Optimum results are obtained where the weight ratio of pamoic acid and sunitinib malate is 1:1, 2:1, 5:1, 8:1, 10:1, 20:1, 30:1 or 40:1.

The resulting microcrystals can be sterilized by washing with ethanol or comparable GRAS alcohol. The size of the microcrystals is between about 1 micron and about 50 microns, more preferably between about 10 micron and about 40 microns, and most preferably between about 20 micron and about 30 microns.

The microcrystals can be formulated into an appropriate excipient for injection into the eye, the ear (especially the inner ear), or neurological tissue, especially the spinal cord or brain.

### BRIEF DESCRIPTION OF THE DRAWINGS

FIGS. 1A and 1B are graphs of the size (length) of the SPC microcrystals after drying. FIG. 1A is a graph of the length of lyophilized SPC microcrystals resuspended in 0.5 mg/mL of CMC, HA, or Poloxamer 407 in water. FIG. 1B is a graph comparing the length of SPC microcrystals after lyophilization or bench top vacuum drying (resuspended after drying with 0.5 mg/mL of CMC for imaging purposes).

### DETAILED DESCRIPTION OF THE INVENTION

### I. Definitions

The term “microcrystal” of an active agent refers to a crystalline form of the agent, typically a synthetic or natural compound having a molecular weight of less than 2000D, more typically less than 1500 or 1000 D, having dimensions between about 0.1 μm and about 1,000 μm, inclusive.

Microcrystals can be either crystalline, amorphous, or a mixture thereof. The term “microcrystal formulation” infers that at least one active agent be present in the formulation is in crystalline form. Typically, the concentration of active agent within a microcrystal is greater than the maximum concentration of active agent in solution. In some embodiments, the concentration of active agent within a microcrystal is between about 1 and about 1,000 mg/ml.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio. “Excipient” is used herein to include a pharmaceutically acceptable compound that is not a therapeutically or biologically active compound. An excipient should generally be inert and non-toxic to the subject.

The terms “biocompatible” and “biologically compatible” generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The terms “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

The terms “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated a disease or disorder of the eye is mitigated or eliminated.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control.

The term “analog” refers to a chemical compound with a structure similar to that of another “reference” compound, but differing from it in respect to a particular component, functional group, atom, etc.

The term “derivative” refers to a compound, which is formed from a parent compound by one or more chemical reaction(s).

### II. Compositions

Compositions including therapeutic or prophylactic agents in micro-crystalline form, particularly DLK inhibitors, enhance and prolong the therapeutic or prophylactic efficacy of the agents within the eye, ear, neurological tissue, or site of neuroinflammation as compared with administration of the agent in non-crystalline form. In preferred embodiments, microcrystals are formed of DLK inhibitors, especially sunitinib, most preferably sunitinib malate in complex with pamoic acid.

### A. Therapeutic and Prophylactic Agents

The compositions contain one or more therapeutic agents or prophylactic agent, to effect or assist in the effecting of a cure or mitigation of a disease or defect, especially in the eye, ear, or neurological tissue. The agent may also be a pro-drug which becomes biologically active or more active when placed in a predetermined physiological environment.

Agents may be small-molecule drugs ((e.g., molecular weight less than 2000, 1500, 1000, 750, or 500 atomic mass units (amu)), peptides or proteins, sugars or polysaccharides, nucleotides or oligonucleotides such as aptamers, siRNA, and miRNA, lipids, glycoproteins, lipoproteins, or combinations thereof.

Agents may be described in Martindale: The Complete Drug Reference, 37^{th} Ed. (Pharmaceutical Press, London, 2011) or others known to those skilled in the art or described in the scientific literature. In one embodiment, the agent to be delivered is poorly soluble in water, and may be administered as a suspension or in a non-aqueous formulation. In other embodiments, the agents are water-soluble.

The microcrystal formulations contain a therapeutically effective amount of a therapeutic agent to treat, inhibit, or alleviate one or more symptoms of a disease state being treated. The compositions can contain an effective amount of a prophylactic agent to prevent one or more symptoms of a disease or disorder in the eye, ear, or nervous tissue, especially brain and spinal cord, particularly for sites that require very low volume and high concentration formulations, including the inner ear.

In preferred embodiments, the active agents are one or more small molecule drugs that are less than about 1500 g/mol, less than about 1000 g/mol, less than about 800 g/mol, or less than about 500 g/mol. In further preferred embodiments, the active agents are one or more small molecule drugs that bind to melanin.

Preferred therapeutic or prophylactic agents are DLK or LZK inhibitors.

Glaucoma, a major cause of blindness worldwide, is a neurodegenerative optic neuropathy in which vision loss is caused by loss of retinal ganglion cells (RGCs). To better define the pathways mediating RGC death and identify targets for the development of neuroprotective drugs, a high-throughput RNA interference screen with primary RGCs was used to identify dual leucine zipper kinase (DLK) as a key neuroprotective target in RGCs. In cultured RGCs, DLK signaling is both necessary and sufficient for cell death. DLK undergoes robust posttranscriptional up-regulation in response to axonal injury in vitro and in vivo. Using a conditional knockout approach, we confirmed that DLK is required for RGC JNK activation and cell death in a rodent model of optic neuropathy. In addition, tozasertib, a small molecule protein kinase inhibitor with activity against DLK, protects RGCs from cell death in rodent glaucoma and traumatic optic neuropathy models. Welsbie, et al. PNAS 110 (10) 4045-4050 (2013) https://doi.org/10.1073/pnas.1211284110.

Chemotherapy-induced peripheral neuropathy (CIPN) and chemotherapy-induced cognitive impairments (CICI) are common, often severe neurotoxic side effects of cancer treatment that greatly reduce quality of life of cancer patients and survivors. The dual leucine zipper kinase (DLK) is a key mediator of axonal degeneration that is localized to axons and coordinates the neuronal response to injury. A brain-penetrant DLK inhibitor, IACS'8287, demonstrates potent and highly selective inhibition of DLK in vitro and in vivo. Co-administration of IACS'8287 with the platinum derivative cisplatin prevents mechanical allodynia, loss of intraepidermal nerve fibers in the hind paws, cognitive deficits, and impairments in brain connectivity in mice, all without interfering with the antitumor activity of cisplatin. Ma, et al, PAIN 162(10): 2599-2612 (2021) DOI: 10.1097/j.pain.0000000000002256.

Traumatic brain injury (TBI) is a major cause of CNS neurodegeneration and has no disease-altering therapies. It is commonly associated with a specific type of biomechanical disruption of the axon called traumatic axonal injury (TAI), which often leads to axonal and sometimes perikaryal degeneration of CNS neurons. Genome scale, arrayed RNA interference-based screens in primary mouse retinal ganglion cells (RGCs) identified a pair of related kinases, dual leucine zipper kinase (DLK) and leucine zipper kinase (LZK) that are key mediators of cell death in response to simple axotomy. Mol Neurodegeneration 14, 44 (2019). https://doi.org/10.1186/s13024-019-0345-1.

In a preferred embodiment, the agent is a DLK inhibitor, most preferably sunitinib, salts, analogues, prodrugs or derivatives thereof. Other DLK inhibitors include tozasertib, GNE3511, GNE8505, GDC-0134, IACS8287, etc. GDC-0134 is a potent, selective, orally available, brain-penetrant small-molecule inhibitor of dual leucine zipper kinase (DLK). GDC-0134 blocks DLK activity in cellular assays and in animal models of neuronal injury (Genentech, data on file). IACS'8287, which demonstrates potent and highly selective inhibition of DLK (Pain 2021 Oct. 1; 162(10): 2599-2612 doi: 10.1097/j. pain.0000000000002256.

### B. Microcrystals of Active Agents

Microcrystals are formed by mixing the agent, preferably in salt form, with an aqueous insoluble agent such as pamoic acid, then precipitating in water to form crystals. The weight ratio is optimized for the individual components.

Excipients used for crystal formation are generally water soluble lipophilic molecules containing anions, like carboxylic acids. Examples other than pamoic acid include sodium oleate, sodium laurate, sodium stearate, and sodium palmitate.

In the most preferred embodiment, the microcrystals are formed by mixing a sunitinib salt such as sunitinib malate with pamoic acid and precipitating in water as a function of the weight ratio of sunitinib salt with pamoic acid. Optimum results are obtained where the weight ratio of pamoic acid and sunitinib malate is 1:1, 2:1, 5:1, 8:1, 10:1, 20:1, 30:1 or 40:1.

In preferred embodiments, compositions and methods useful for in vivo delivery of one or more active agents, in the form of microcrystals that are suitable for parenteral administration in aqueous particulate suspension. In further preferred embodiments, the formulations including microcrystals free of non-crystalline aggregates. The microcrystal suspension is typically suited for administration by 25-, 27- or 30-gauge needles.

### C. Pharmaceutical Formulations

Pharmaceutical compositions including microcrystals of one or more active agents such as sunitinib and pamoic acid may be formulated in a conventional manner using one or more physiologically acceptable carriers including excipients and auxiliaries which facilitate processing of the microcrystals into preparations which can be used pharmaceutically.

Formulation is dependent upon the route of administration. In preferred embodiments, the compositions are formulated for parenteral delivery. In some embodiments, the compositions are formulated for intraocular injection. Typically, the compositions will be formulated in an isotonic solution to the eye, about 300 mOsm/L, for example, in sterile saline or buffered solution for injection into the tissues or cells to be treated in the eye, ear, or neurological tissue such as the spinal cord or brain. The compositions can be stored frozen or lyophilized in single use vials for rehydration immediately before use. Other means for rehydration and administration are known to those skilled in the art.

Pharmaceutical formulations contain one or more microcrystals of active agents for treating or preventing eye, ear or neuroinflammation diseases or disorders in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably materials which are generally recognized as safe (GRAS), and may be administered to an individual without causing undesirable biological side effects or unwanted interactions. The pharmaceutical excipients should not reduce the stability of the microcrystals.

### III. Method of Making Organic Solvent Free Microcrystals

In the most preferred embodiment, the microcrystals are formed by mixing sunitinib salt such as sunitinib malate with pamoic acid and precipitating in water as a function of the weight ratio of sunitinib salt with pamoic acid.

Optimum results are obtained where the weight ratio of pamoic acid and sunitinib malate is 1:1, 2:1, 5:1, 8:1, 10:1, 20:1, 30:1 or 40:1. See Example 1.

The resulting microcrystals can be sterilized by washing with ethanol or comparable GRAS alcohol. The size of the microcrystals is between about 1 micron and about 50 microns, more preferably between about 10 micron and about 40 microns, and most preferably between about 20 micron and about 30 microns. Other factors can also impact size, such as stirring rate and temperature, as well as shape. In the preferred embodiment the microcrystals are predominantly needle shaped, although they may also be rod shaped, or a mixture thereof.

### IV. Methods of Use

The compositions of microcrystals are preferably formulated in dosage unit form for ease of administration and uniformity of dosage. The phrase “dosage unit form” refers to a physically discrete unit of conjugate appropriate for the patient to be treated. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions of microcrystals are administered locally, for example, by injection directly into a site to be treated. In some embodiments, the compositions are injected, topically applied, or otherwise administered directly into the eye, inner ear or brain or spinal cord. Typically, the microcrystals are injected as a suspension in solution, in a volume of between about 0.1 μl and about 1,000 μl, preferably between about 1 μl and 50 μl. In a particular embodiment, the methods deliver microcrystals of Sunitinib malate conjugated to pamoic acid by intravitreal, sub-conjunctival, or supra-choroidal injection into the eye for treatment and prevention of an eye disease, such as glaucoma. The formulation may be administered into other tissues where a small volume is required, such as the inner ear, and/or sites of neuroinflammation, such as the spinal cord and brain.

In some examples, the compositions of microcrystals retain an effective concentration of one or more active agents at or near the site of administration and/or the target site for an extended period of time, for example, more than 1 day, more than 2 days, more than 3 days, more than 4 days, more than 5 days, more than 6 days, or more than a week. In preferred cases, the compositions of microcrystals are administered once a week or less frequently, once every other week or less frequently, once every month or less frequently, once every other month or less frequently The present invention will be further understood by reference to the following non-limiting examples.

### EXAMPLES

### Example 1: Microcrystallization of Sunitinib Malate Without Organic Solvent

### Materials and Methods

### Material Sources

Sunitinib malate was purchased from LC Laboratories. Pamoic acid disodium salt was purchased from Tokyo Chemical Industry. Endotoxin-free water, 50 mL Falcon conical tubes, 50 mL glass scintillation vials, and PTFE magnetic stir bars (5 mm-3 cm) were purchased Fisher Scientific. FreezeCell™ Circular Shape for Eppendorf, isopropyl alcohol, sodium carboxymethyl cellulose (CMC, 173 kDa), hyaluronic acid (HA, 2 MDa), and Poloxamer 407 (407, 12.6 kDa) were purchased from Sigma Aldrich. PluriStrainers (1 μm) with connector ring were purchased from PluriSelect.

### SPC Synthesis and Characterization

Sunitinib malate (240 mg) was dissolved in endotoxin-free water (60 mL) at 4 mg/mL in a 50 mL Falcon conical tube or a 50 mL glass scintillation vial with the aid of probe sonication (40% energy intensity, 1 s/1 s on/off cycle for 60 min at room temperature). Pamoic acid disodium salt was dissolved in endotoxin-free water at various concentrations (4, 8, 20, 32, 40, 80, 120, and 160 mg/mL) in a 50 mL Falcon conical tube or 50 mL scintillation vial. which corresponded to the following weight ratios of pamoic acid disodium salts to sunitinib malate Equal volumes (5, 10, 15, 20 and 25 mL) of 4 mg/mL sunitinib and the various concentrations of pamoic acid disodium salt solution was mixed together to achieve various mass ratios (1:1, 2:1, 5:1, 8:1, 10:1, 20:1, 30:1, 40:1 pamoic acid:sunitinib malate). Mixing of solutions was achieved with various sized PTFE magnetic stir bars (5 mm-3 cm) and stirring speeds (50-600 rpm). The mixtures were then placed on an orbital rotator at various speeds (10, 15, 20 and 25 rpm). All mixtures were incubated at various temperatures (4 °C, 21 °C, 30 °C, 40 °C, 45 °C, 50 °C). After 2, 4, 6, 18, or 24 hours of mixing, 5 μL of the suspension were collected from the solution and placed on a glass slide to observe the morphology of the precipitate at 40× on a bright field microscope.

The SPC microcrystals were collected and washed three times with endotoxin-free water. To sterilize the rod-shaped SPC microcrystals, the crystals (10 mg) were transferred to a 1 μm filter mesh and submerged in 1 mL of 70% isopropyl alcohol for 60 seconds. Bench top vacuum filtration was used to remove the isopropyl alcohol. The SPC microcrystals were then collected for slow freezing with a FreezeCell™ in a −80 °C freezer before lyophilization. The SPC microcrystal samples were lyophilized for 48 hours to completely remove residual water. Alternatively, the crystals were centrifuged at 2000 g for 10 minutes to remove as much water as possible, and dried with bench top vacuum for 48 hours. Solutions containing 0.5 mg/mL CMC, HA, and Poloxamer 407 were used to resuspend the dried crystals before imaging with bright field microscope using 20× object lens. The dimensions of at least 10 microcrystals per sample were measured with Nikon imaging software (1.07v). To measure the drug loading, the dried SPC microcrystals were weighed and dissolved with DMSO. The sunitinib concentration was measured using a fluorescence standard curve at λₑₘ=540 (n=3).

### Results

Representative images were made of SPC aggregates/particulates with various processing conditions. SPC microcrystals were suspended in 0.5 mg/mL CMC for imaging purposes. SPC formed needle-shape aggregates with lower weight ratios (1:1-10:1) of pamoic acid: sunitinib malate, or generated few rod-shaped SPC crystals dominated by needle-shape aggregates. Higher complexation ratios, including 20:1 and 30:1 incubated at 40-50 °C, volume <20 mL, and rotation speed >20 rpm, yielded a higher proportion of rod-shaped SPC crystals. The ideal SPC microcrystal processing conditions utilized 40:1 complexation ratio, 45 °C mixing temperature, 20 rpm rotation speed, 20 mL volume, and 24 hours incubation time.

It was discovered that both the speed of the rotational mixing and the volume of fluid had a significant effect on the crystal formation. Too low of mixing speed (e.g. <10 rpm) and high mixture volume (>25 mL) led to amorphous aggregates on the walls of the container because of insufficient fluid mixing inside the tube. More aggregation was observed using glass vials, so polypropylene conical tubes were used moving forward. Further, it was surprisingly observed that the weight ratio of pamoic acid to sunitinib malate and the mixing temperature had large effects on the morphology of the SPC aggregates and particulates that formed. Mixing pamoic acid and sunitinib malate at 1:1, 2:1, 5:1, 8:1 and 10:1 weight ratio resulted in needle-shape SPC aggregates and few rod-shaped morphology SPC crystals. However, the needle-shape aggregates dominated the overall populations of SPC. Higher pamoic acid: sunitinib malate ratios, including 20:1 and 30:1 in a total volume <20 mL incubated at 40 °C, 45 °C and 50 °C at a rotation speed >20 rpm yielded a higher proportion of the rod-shaped SPC crystals, though the needle-shape aggregates were still abundant in the samples ). The highest yield of monoclinic, rod-shaped SPC microcrystals was obtained using a 40:1 pamoic acid: sunitinib malate ratio at 45 °C, 20 rpm rotation speed, 20 mL volume, and 24 hours incubation. The average drug loading as a percentage of the total mass was found to be 50.4±2.4%, and the yield was 45.0±4.4%.

The crystals could be further sterilized with 70% isopropyl alcohol and remained intact and of similar size after lyophilization or vacuum drying. For the purpose of imaging the microcrystals to measure the dimensions, various polymers at 0.5 mg/mL were used to resuspend the microcrystals.

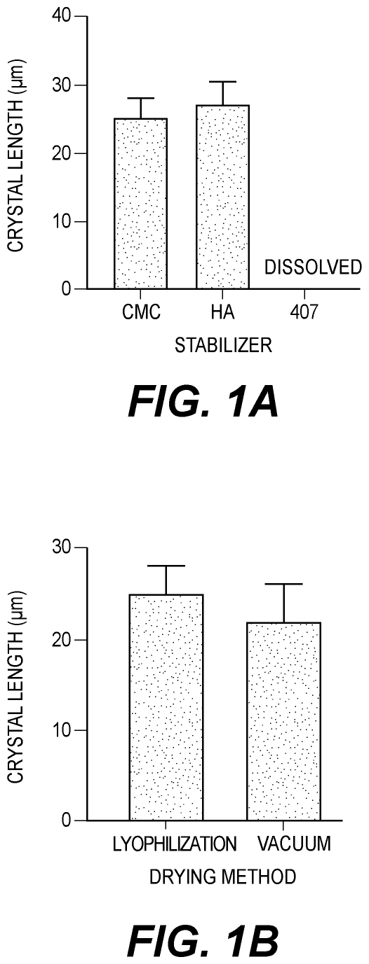

Resuspension yielded sizes similar to the starting dimensions before lyophilization using either CMC (length 25±3.1 μm, width 16±1.4 μm) or HA (27±3.4 μm, width 16±1.5 μm) (FIG. 1A). In contrast, Poloxamer 407 dissolved the SPC microcrystals. Both drying processes did not affect the rod-shaped morphology SPC crystals. Resuspension of SPC microcrystals in CMC after bench top vacuum drying also yielded similar dimensions (length 22±4.1 μm, width 15±1.9 μm) (FIG. 1B).

Variations and modifications of the foregoing will be obvious to those skilled in the art and are intended to come within the scope of the appended claims.

1. Organic solvent free drug microcrystals, wherein the drug is a therapeutic or prophylactic agent for treatment of the eye, ear, or neurological tissue.

2. The microcrystals of claim 1, wherein the one or more therapeutic agents or prophylactic agent bind to a ligand or receptor in or on the eye or is an inhibitor of dual leucine zipper kinase (DLK) or leucine zipper kinase (LZK).

3. The microcrystals of claim 2, wherein the one or more therapeutic agents or prophylactic agents is sunitinib, tozasertib, GNE3511, GNE8505, GDC-0134, IACS8287, salts, derivatives, analogues, or prodrugs thereof.

4. The microcrystals of claim 3 wherein the agent is combined with a water soluble lipophilic compound containing anions selected from the group consisting of carboxylic acids, pamoic acid, sodium oleate, sodium laurate, sodium stearate, and sodium palmitate and precipitated in water. water soluble lipophilic molecules containing anions, like carboxylic acids.

5. The microcrystals of claim 3, wherein the microcrystals are formed by mixing a sunitinib salt with pamoic acid and precipitating in water as a function of the ratio of sunitinib salt with pamoic acid.

6. The microcrystals of claim 5, wherein the microcrystals are formed of sunitinib malate and pamoic acid.

7. The microcrystals of claim 6 wherein the weight ratio of pamoic acid and sunitinib malate is 1:1, 2:1, 5:1, 8:1, 10:1, 20:1, 30:1 or 40:1.

8. The microcrystals of claim 1, wherein the size of the microcrystals is between about 1 micron and about 50 microns.

9. The microcrystals of claim 1, wherein the size of the microcrystals is between about 10 micron and about 40 microns.

10. The microcrystals of claim 1, wherein the size of the microcrystals is between about 20 micron and about 30 microns.

11. The microcrystals of claim 1, formulated for intravitreal, subconjunctival, or suprachoroidal administration.

12. The microcrystals of claim 1, wherein the microcrystals are predominantly needle shaped.

13. The microcrystals of claim 1, in a formulation for administration to the eye, ear, or neurological tissue such as the spinal cord or brain.

14. A method for preventing and treating one or more diseases, conditions, or injuries of the eye, ear or neurological tissue in a subject comprising administering to the eye, ear, neurological tissue or sites of neuroinflammation of the subject a therapeutically effective amount of a pharmaceutically acceptable formulation comprising the microcrystals of claim .

15. The method of claim 14, wherein the microcrystal formulation is administered to the eye or a compartment thereof.

16. The method of claim 14, wherein the microcrystal formulation is administered to the ear or a compartment thereof.

17. The method of claim 14, wherein the microcrystal formulation is administered to neurological tissue, such as the spinal cord or brain, or neuroinflammation associated with chemotherapy.
